(19) United States
(12) Patent Application Publication
(10) Pub. No.: US 2010/0121189 A1
(43) Pub. Date: May 13, 2010

(54) SYSTEMS AND METHODS FOR IMAGE PRESENTATION FOR MEDICAL EXAMINATION AND INTERVENTIONAL PROCEDURES

(75) Inventors: Qinglin Ma, Woodinville, WA (US); Nikolaos Pagoulatos, Bothell, WA (US)

Correspondence Address: SonoSite, Inc. / Fulbright & Jaworski, L.L.P., 2200 Ross Avenue, Suite 2800, Dallas, TX, 75201, US

(73) Assignee: Sonosite, Inc., Bothell, WA (US)

(21) Appl. No.: 12/269,623

(22) Filed: Nov. 12, 2008

(51) Int. Cl. A61B 8/00; G06K 9/00

(52) U.S. Cl. 600/437; 382/131

(57) ABSTRACT

Systems and methods which provide image presentation for medical examination, interventional procedures, diagnosis treatment, etc. from multi-dimensional volume datasets are shown. Reference indicators, providing information with respect to the relationship of an image to the physical world, are preferably provided to aid a viewer in interpreting the image. Such reference indicators may be provided in the form of tool markers and corresponding image marker. Degrees of freedom provided with respect to image manipulation are preferably selectively constrained to facilitate interaction with an image or images. Embodiments of the invention may implement a relatively simple bidirectional control to facilitate a survey of an entire image volume. Image display conventions may be provided which present images in a particular orientation to facilitate user interpretation of the image.

### REFERENCE TO RELATED APPLICATIONS

The present application is related to co-pending and commonly assigned U.S. patent application Ser. No. [Attorney Docket No. 65744-P044US-10805628] entitled “Systems and Methods to Identify Interventional Instruments,” filed concurrently herewith the disclosure of which is hereby incorporated herein by reference.

### TECHNICAL FIELD

The invention relates generally to image presentation and, more particularly, to image presentation for medical examination, interventional procedures, diagnosis, treatment, etc.

### BACKGROUND OF THE INVENTION

Various forms of imaging apparatus have been used extensively for medical applications. For example, fluoroscope systems, X-ray imaging systems, ultrasound imaging systems, computed tomography (CT) imaging systems, and magnetic resonance (MR) imaging (MRI) systems have been used for a number of years. For example, medical examination, interventional procedures, diagnosis, and/or treatment may be provided using an appropriate one of the foregoing systems suited for the task.

Images provided by imaging apparatus such as fluoroscope systems, X-ray imaging systems, and ultrasound imaging systems had traditionally been two-dimensional (2D) (e.g., a planar image providing information in an X and Y axes space). For example, fluoroscope systems and X-ray imaging systems traditionally provide a 2D image of a target broadside shadow on an image receptor. Ultrasound imaging systems, on the other hand, traditionally provided a 2D cross-sectional view of a portion of an ensonified target. Marking systems have been used, wherein a transducer is provided with a marker and a dot is displayed in a corresponding image, for associating left-right in the ultrasound image with the transducer.

Computing technology, having progressed dramatically in the last few decades, has provided three-dimensional (3D) (e.g., a planar image providing information in an X, Y, and Z axes space) and even four-dimensional (4D) (e.g., a 3D image having a time axis added thereto) imaging capabilities. Although such 3D and 4D imaging technology arose from disciplines such as drafting, modeling, and even gaming, the technology has been adopted in the medical field. For example, computed tomography has been utilized with respect to X-ray images to produce 3D images. Furthermore, computerized 3D rendering algorithms have been utilized to enhance the visualization of 3D datasets from various imaging modalities including CT, MR, ultrasound etc.

The use of such computing technology to provide 3D and 4D images in the medical field has carried with it several disadvantages from its origins. For example, it has typically been considered an advantage of such computing technology to provide a large number of degrees of freedom with respect to the rendered images. Specifically, from the drafting and modeling roots of 3D imaging, it has been believed that providing bi-axial freedom of movement/rotation with respect to each of the X, Y, and Z axes (i.e., 6 degrees of freedom). Such degrees of freedom can be used to allow 2D cross-section images through a 3D volume (e.g., multi-planar reconstruction (MPR) images) in any plane. Particular orientations, such as top, bottom, left, and right, are often less important in the virtual world than presenting a desired portion of the rendered image to a viewer. Accordingly, object image (e.g., volume rendered (VR) image) and cross-section image (e.g., MPR image) orientation freedom has been provided by 3D and 4D image computing technology.

Providing such freedom with respect to certain medical imaging tasks has been acceptable and even useful. For example, when imaging a fetus or a heart, whose landmark structures are readily recognizable, providing object image and cross-section image freedom is not problematic because the person examining the image is able to easily determine the proper orientation of the target mentally due to familiarity with the shape of the target. Moreover, because the viewer is examining the structure, rather than performing some form of interventional procedure, such freedom in displaying the image can facilitate the examination.

However, the present inventors have discovered that when imaging less recognizable structure and/or utilizing images for interventional procedures, such image display freedom can lead to an inability to interpret the image and confusion by the person examining the image. For example, when the target includes less recognizable structure, such as nerves, intestines, tumors, etc., the orientation of the object or cross-section may be paramount to identifying the structure within the image. Where a technician, such as a physician, is attempting an interventional procedure, such as inserting a needle or catheter in a patient to achieve a precise placement, such freedom in displaying the image can result in confusion and an inability to determine the correct movements to be made. Accordingly, the use of 3D and 4D medical imaging has heretofore been limited in its applicability.

### BRIEF SUMMARY OF THE INVENTION

The present invention is directed to systems and methods which provide image presentation for medical examination, interventional procedures, diagnosis, treatment, etc. from multi-dimensional (e.g., three-dimensional and/or four-dimensional) volume datasets in a manner adapted to facilitate readily interpreting the image and/or performing the desired task. One or more reference indicator, providing information with respect to the relationship of an image to the physical world, is preferably provided to aid a viewer in interpreting the image in accordance with concepts of the present invention. Degrees of freedom provided with respect to image manipulation are preferably selectively constrained to facilitate interaction with an image or images and/or to aid the viewer in understanding the image.

Embodiments of the invention provide one or more reference indicator in the form of a marker or markers to correlate sides, dimensions, etc. of an image volume dataset with the physical world. For example, a tool, such as an ultrasonic transducer, may be provided with tool markers useful for correlating sides of the tool with sides or dimensions of images generated using the tool. According to an embodiment of the invention, a tool marker having a first attribute (e.g., color, shape, sound, texture, etc.) and a tool marker having a second attribute (e.g., a different color, shape, sound, texture, etc.) are provided on selected sides of the tool. Correspondingly, an image marker having a portion with the first attribute and a portion with the second attribute is provided in association with a rendered image to thereby provide an intuitive guide for a viewer to recognize and understand the orientation of the image in relationship to the tool used to generate the image. According to embodiments of the invention, the image marker comprises a representational pictogram providing orientation, spatial, and/or relational information.

Embodiments of the invention may implement any number of tool markers and image markers as determined to provide desired correlation of image to physical space. Moreover, the number of tool markers and image markers in any particular embodiment may be different (as in the embodiment described above) or the same (such as to provide a first and second tool marker and a first and second image marker). The concepts of the invention are not limited to use of tool and/or image markers. For example, physical space markers may be implemented in combination with image markers to provide correlation of image to physical space.

One or more image display convention may be selected for use in imaging provided with respect to particular tasks, uses, situations, etc. For example, where imaging is performed with respect to structure which is not easily recognizable and/or for interventional procedures, an image display convention may be utilized according to embodiments of the invention to facilitate interpretation of the image by a person examining the image. Such image display conventions may include an image coordinate system for always presenting an image in a particular orientation, such as to always orient shallow up and deep down in an image generated by an ultrasound system, irrespective of the orientation of the image plane within a volume being imaged.

Embodiments of the invention additionally or alternatively restrict the numbers and types of images which may be displayed. According to embodiments of the invention, the degrees of freedom for image plane rotation about various axes of a multi-dimensional volume, such as when selecting an image plane for generating images. For example, although 6 degrees of freedom (e.g., bidirectional rotation about the X axis, bidirectional rotation about the Y axis, and bi-directional rotation about the Z axis) are often available with respect to a 3D volume, embodiments of the present invention impose restrictions to the degrees of freedom with respect to image plane selection. According to an embodiment of the invention, the ability to generate MPR images from a multi-dimensional image volume are limited to cross-sectional images generated along a particular axis, arc, etc. The use of image plane freedom limitations according to embodiments of the invention synergistically is accompanied by a simplified user interface. For example, where the ability to generate MPR images is limited to cross-sectional images generated along an axis or arc, embodiments of the invention may implement a relatively simple bidirectional control, such as left and right buttons, spinner knob, single axis joystick, etc. Moreover, the use of image plane freedom limitations according to embodiments of the invention preferably facilitates a user's ability to obtain an image or images needed or desired for a particular task or use, as well as preventing a user from accidentally failing to obtain a needed or desired image.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1A shows a system adapted according to an embodiment of the present invention;

FIG. 1B shows a high level block diagram of an embodiment of the system of FIG. 1A;

FIGS. 2A and 3A show exemplary images as may be generated by the system of FIG. 1A;

FIGS. 2B and 3B show the image planes of a respective one of the images of FIGS. 2A and 3A;

FIGS. 4A-4C show an exemplary embodiment of a pictogram as may be displayed as an image marker by the system of FIG. 1A;

FIG. 5 shows an exemplary image as may be generated to include various sub-images by the system of FIG. 1A; and

FIGS. 6A and 6B show how an image display convention may be implemented mathematically according to embodiments of the invention.

### DETAILED DESCRIPTION OF THE INVENTION

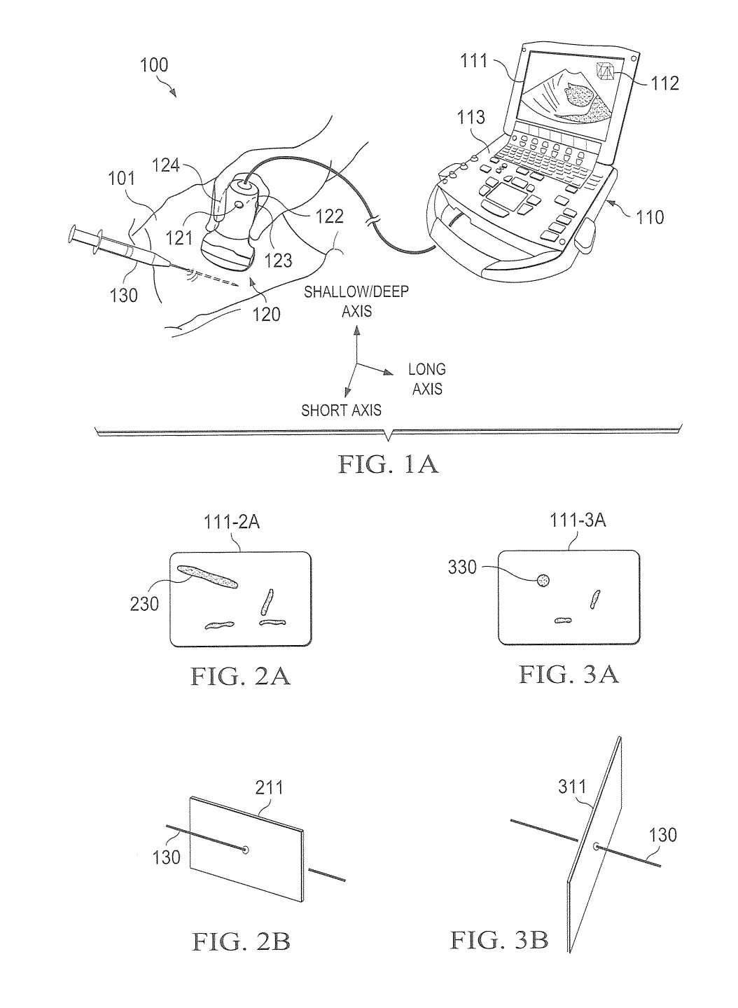

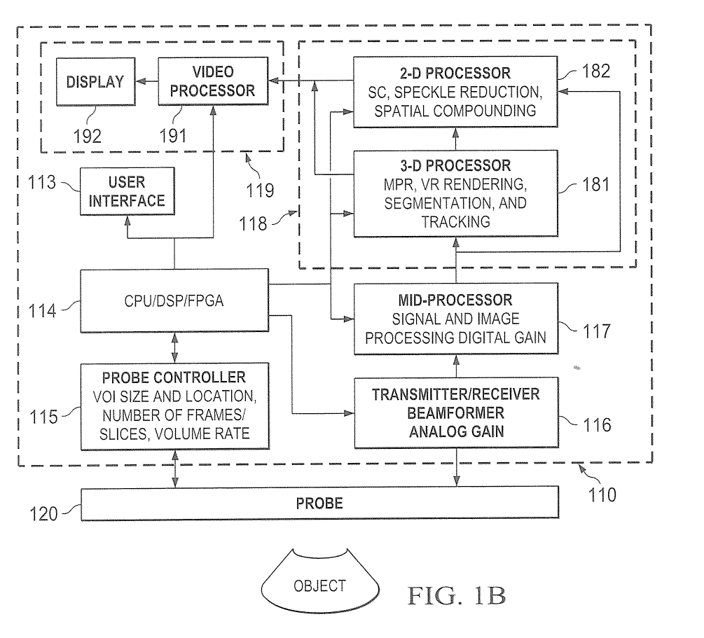

Directing attention to FIG. 1A, a system adapted according to embodiments of the invention is shown as system 100. System 100 may, for example, comprise a diagnostic ultrasound system operable to provide 2D and/or 3D images from a multi-dimensional (e.g., 3D and/or 4D) volume dataset. Although embodiments of the invention are described herein with reference to ultrasound imaging technology, in order to aid the reader in understanding the invention, it should be appreciated that the concepts of the present invention are not limited in applicability to ultrasound imaging. For example, embodiments of the present invention may be implemented with respect to fluoroscope systems, X-ray imaging systems, ultrasound imaging systems, CT imaging systems, MRI systems, positron emission tomography (PET) imaging systems, and the like.

System 100 of the illustrated embodiment includes system unit 110 and transducer 120 coupled thereto. System unit 110 preferably comprises a processor-based system, such as shown in the high level block diagram of FIG. 1B. Transducer 120 may comprise a transducer configuration corresponding to the imaging technology used.

System unit 110 illustrated in FIG. 1B includes processor 114, such as may comprise a central processing unit (CPU), digital signal processor (DSP), field programmable gate array (FPGA), and/or the like, preferably having memory associated therewith. In embodiments, the processor-based system of system unit 110 may comprise a system on a chip (SOC), for example. Probe controller 115 of system unit 110 shown in FIG. 1B provides image dataset collection/acquisition control, such as to control the volume of interest size and location, the volume rate, the number of imaging slices used for image acquisition, etc. Front-end circuitry 116 of the illustrated embodiment provides signal transmission to drive probe 120, beamforming for transmission and/or reception of ultrasonic pulses, signal conditioning such as filtering, gain control (e.g., analog gain control), etc. Mid-processor 117 of the illustrated embodiment, operable under control of processor 114, provides signal and image processing, additional signal conditioning such as gain control (e.g., digital gain control), decimation, low-pass filtering, demodulation, re-sampling, lateral filtering, compression, amplitude detection, blackhole filling, spike suppression, frequency compounding, spatial compounding, decoding, and/or the like.

According to the illustrated embodiment, signals processed by mid-processor 117 are provided to back-end processor 118 for further image processing. Back-end processor 118 of the illustrated embodiment includes 3D processor 181 and 2D processor 182. 3D processor 181, operating under control of processor 114, produces 3D image volumes and images therefrom (e.g., MPR images, VR images) for presentation by display system 119 as image 111. 3D processor 181 of the illustrated embodiment further provides for image volume segmentation, image plane determination, interventional instrument tracking, gray mapping, tint mapping, contrast adjustment, MPR generation, volume rendering, surface rendering, tissue processing, and/or flow processing as described herein. 2D processor 182, operating under control of processor 114, provides scan control, speckle reduction, spatial compounding, and/or the like.

User interface 113 of embodiments may comprise keyboards, touch pads, touch screens, pointing devices (e.g., mouse, digitizing tablet, etc.), joysticks, trackballs, spinner knobs, buttons, microphones, speakers, display screens (e.g., cathode ray tube (CRT), liquid crystal display (LCD), organic LCD (OLCD), plasma display, back projection, etc.), and/or the like. User interface 113 may be used to provide user control with respect to multi-dimensional image mode selection, image volume scanning, object tracking selection, depth selection, gain selection, image optimization, patient data entry, image access (e.g., storage, review, playback, etc.), and/or the like. Display system 119, comprising a part of user interface 113 of embodiments of the invention, includes video processor 191 and display 192. Video processor 191 of embodiments provides video processing control such as overlay control, gamma correction, etc. Display 192 may, for example, comprise the aforementioned CRT, LCD, OLCD, plasma display, back projection display, etc.

Logic of system unit 110 preferably controls operation of system 100 to provide various imaging functions and operation as described herein. Such logic may be implemented in hardware, such as application specific integrated circuits (ASICs) or FPGAs, and/or in code, such as in software code, firmware code, etc.

According to a preferred embodiment, transducer 120 comprises one or more transducer elements (e.g., an array of ultrasound transducers) and supporting circuitry to illuminate (e.g., insonify) a target, capture data (e.g., ultrasound echos), and provide target data (e.g., transducer response signals) to system unit 110 for use in imaging. Transducer 120 of the embodiment illustrated in FIG. 1B may, for example, comprise any device that provides conversion between some form of energy and acoustic energy, such as a piezoelectric transducer, capacitive micro-machined ultrasonic transducer (CMUT), a piezoelectric micro-machined ultrasonic transducer (PMUT), etc. Where system unit 110 comprises an ultrasound imaging system unit, transducer 120 may comprise any of a number of ultrasound transducer configurations, such as a wobbler configuration, a 1D matrix array configuration, a 1.5D matrix array configuration, a 1.75D matrix array configuration, a 2D matrix array configuration, a linear array, a curved array, etc. Moreover, transducer 120 may be adapted for particular uses, procedures, or functions. For example, transducers utilized according to embodiments of the invention may be adapted for external use (e.g., topological), internal use (e.g., esophageal, vessel, rectal, vaginal, surgical, etc.), cardio analysis, OB/GYN examination, etc.

It should be appreciated that, although the embodiment illustrated in FIG. 1B shows one particular division of functional blocks between system unit 110 and transducer 120, various configurations of the division of functional blocks between the components of system 100 may be utilized according to embodiments of the invention. For example, beamformer 116 may be disposed in transducer 120 according to embodiments of the invention.

In the illustrated embodiment, system 100 is being used with respect to an interventional procedure. Specifically, transducer 120 is being held against object 101, such as may comprise a portion of a human body, to illuminate an area targeted for an interventional procedure. Interventional apparatus 130, such as may comprise a hypodermic needle, a catheter, a portacath, a stent, an intubation tube, endoscope, etc., is being inserted into object 101 in an area illuminated by transducer 120. Accordingly, an image, shown as image 111, is generated by system unit 110 in an effort for a user to visually monitor the progression, placement, and/or use of interventional apparatus 130.

System unit 110 may provide various signal and/or image processing techniques in providing image 111, such as tissue harmonic imaging (THI), demodulation, filtering, decimation, interpretation, amplitude detection, compression, frequency compounding, spatial compounding, black hole fill, speckle reduction, etc. Image 111 may comprise various forms or modes of images, such as color images, B-mode images, M-mode images, Doppler images, still images, cine images, live images, recorded images, etc.

In operation according to traditional imaging techniques, it is often difficult for a user to effectively utilize a system such as system 100 with respect to an interventional apparatus. For example, it is often a cumbersome process to obtain a desirable image plane to show interventional apparatus. Moreover, the orientation of an image plane provided from a 3D image volume is often oriented in a way that is not readily understood. Directing attention to FIG. 2A, image 111-2A is shown presenting a 2D imaging plane (imaging plane 211 of FIG. 2B) along the long axis of transducer 120 (e.g., an ultrasound transducer array longitudinal axis). Similarly, image 111-3A of FIG. 3A presents a 2D imaging plane (imaging plane 311 of FIG. 3B) along the short axis of transducer 120 (e.g., an ultrasound transducer array latitudinal axis).

The longitudinal axis of interventional apparatus 130 is in a plane oriented at a slightly different angle than that of imaging plane 211. Thus, only a relatively small portion of interventional apparatus 130 is visible in image 111-2A as object 230. The longitudinal axis of intervention apparatus 130 is in a plane oriented at an acute angle with respect to that of imaging plane 311. Thus, even a smaller portion of interventional apparatus 130 is visible in image 111-3A as object 330. These relatively small portions of interventional apparatus 130 may not provide visualization of all relevant or desired portions of interventional apparatus 130, such as a distal end, an operative portion, etc. Accordingly, a user is unlikely to be provided with desired information with respect to an interventional procedure from either image 111-2A or 11-3A. Moreover, it is often difficult, if not impossible, for a user to manipulate a transducer with sufficient precision to generate an image providing desired information with respect to an interventional procedure. For example, experience has shown that it is unproductively difficult to attempt to manipulate an ultrasound transducer, providing a 2D planar view of a target area, sufficiently to capture the length of an interventional apparatus within an image. This is often referred to as hand/eye coordination difficulty.

It should be appreciated that typical 3D or 4D imaging technology may not fully address the need with respect to providing imaging in association with interventional procedures. For example, although transducer 120 may be utilized to generate a multi-dimensional (e.g., 3D or 4D) volume dataset from which a volume rendered image may be generated, display of a 3D or 4D object image may not readily convey the needed or desired information. Although an object of interest may be contained in the volume dataset, it may require considerable manipulation to find an image plane that shows the object in a meaningful way. Moreover, where freedom of movement/rotation with respect to each of the X, Y, and Z axes is allowed, the user may not be able determine the orientation of the objects represented, and thus be unable to identify a target object or other objects of interest.

It is often desirable to view both the anatomy and the interventional instrument. Accordingly, a volume rendered image or surface rendered image alone generally does not provide the best fit. Although MPR images, as may be rendered from a multi-dimensional volume dataset generated using transducer 120, comprise 2D images and thus may be used to present an image format that is more readily interpreted by a user and which are suited for display on a 2D output device, control of system unit 110 to display a desired MPR image often proves unproductively difficult. For example, a user may desire to generate a MPR image for an image plane corresponding to the longitudinal axis of interventional apparatus 130 from a multi-dimensional dataset. However, controlling system unit 110 to identify that image plane, such as through input of pitch, yaw, and roll control, may be quite complicated. Moreover, the degrees of freedom available to the user may result in an inability for the user to identify a best MPR image (e.g., the user may be presented with the ability to generate so many variations of images that the best image may never be arrived at). Once generated, the user may be unable to determine the orientation of the image and/or objects therein (e.g., the target object) and thus may be unable to meaningfully interpret the image.

Embodiments of the present invention facilitate ready interpretation of the image and/or performance of desired tasks by providing a plurality of reference indicators in the form of a marker or markers to correlate sides, dimensions, etc. of an image volume dataset with the physical world. In the embodiment illustrated in FIG. 1A, transducer 120 is provided with tool markers 121-124 (it being understood that tool marker 122 of the illustrated embodiment is disposed in a location on the back side of transducer 120 corresponding to that of tool marker 121 shown on the front side of transducer 120, and tool marker 124 of the illustrated embodiment is disposed in a location on the left side of transducer 120 corresponding to that of tool marker 123 shown on the right side of transducer 120) useful for correlating sides of the tool with sides or dimensions image 111 generated using transducer 120. Correspondingly, image marker 112 is provided in, or in association with, image 111 to provide correlating information with respect to a plurality of tool markers 121-124.

According to an embodiment of the invention, tool marker 121 may comprise a first color (e.g. red), tool marker 122 may comprise a second color (e.g., blue), tool marker 123 may comprise a third color (e.g., green), and tool marker 124 may comprise a fourth color (e.g., yellow) so as to provide readily distinguishable attributes in association with a plurality of sides of transducer 120. Of course, additional or alternative marker attributes, such as shape, sound, texture, etc., may be utilized to provide distinguishable attributes in association with sides of transducer 120. Moreover, tool markers may be utilized with respect to different physical attributes of a tool, such as additional or alternative sides (e.g, top, bottom, etc.), physical attributes (e.g., longitudinal axis, latitudinal axis, etc.), and the like.

Although the illustrated embodiment of transducer 120 includes 4 tool markers it should be appreciated that embodiments of the present invention may comprise a different plurality of tool markers. Preferred embodiments of the invention comprise a plurality of tool markers which include at least 2 tool markers associated with orthogonal attributes of an imaging tool, such as different axes. As an example, an embodiment of the present invention may comprise tool marker 121 corresponding to a first axis of transducer 120 and tool marker 123 corresponding to a second axis of transducer 120.

Image marker 112 of embodiments of the invention has a portion with the first tool marker attribute (e.g., red color) and corresponding to a first side (e.g., front side as indicated by tool marker 121) of transducer 120 and a portion with the second tool marker attribute (e.g., green color) and corresponding to a second side (e.g., right side as indicated by tool marker 123) of transducer 120. By providing image marker 112 in association with a rendered image, an intuitive guide is provided for use in recognizing and understanding the orientation of image 111 in 3D relationship to transducer 120. That is, the portion of image marker 112 provided in the first color corresponds to tool marker 121 and the first side of transducer 120 and the portion of image marker 112 provided in the second color corresponds to tool marker 123 and the second side of transducer 120. Accordingly, when a user views image 111, having image marker 112 provided in association therewith (e.g., superimposed on the image itself, displayed in association with the image, etc.), the user may readily recognize the orientation of the image in 3D space as it relates to the orientation of transducer 120.

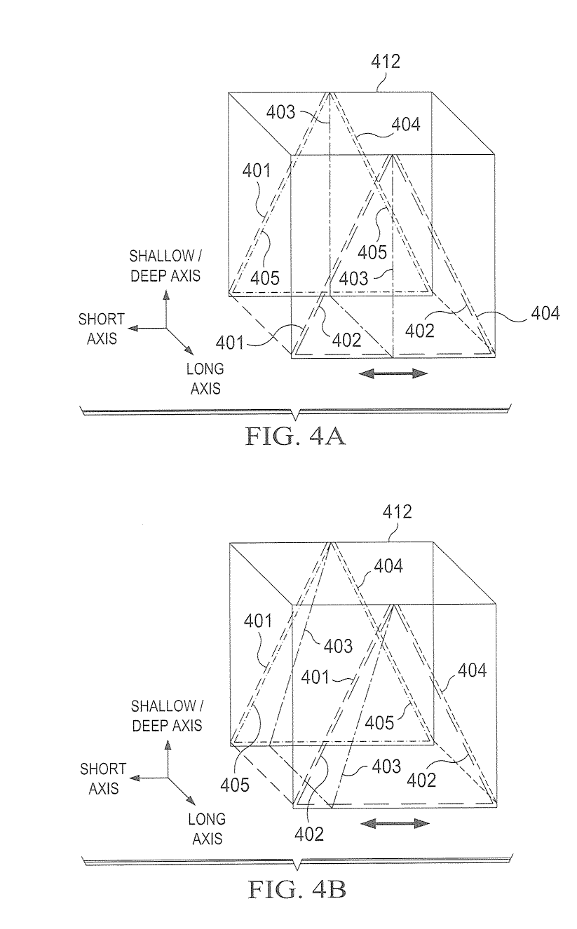

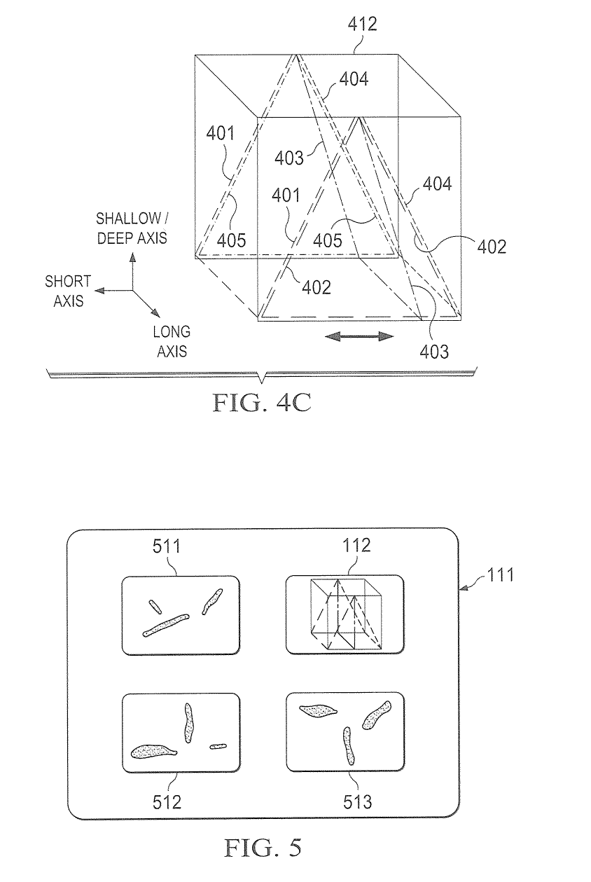

Image marker 112 of embodiments comprises a representational pictogram providing orientation, spatial, and/or relational information. Directing attention to FIG. 4A, an exemplary embodiment of a pictogram as may be utilized according to embodiments of the invention is shown as pictogram 412. Pictogram 412 of the illustrated embodiment includes portion 401 provided in a first color (shown here as a first dotted line pattern) corresponding to a color of tool marker 121, portion 402 provided in a second color (shown here in a second dotted line pattern) corresponding to a color of tool marker 123, portion 404 provided in a third color (shown here in a third dotted line pattern) corresponding to a color of tool marker 122, and portion 405 provided in a fourth color (shown here in a fourth dotted line pattern) corresponding to a color of tool marker 124. According to an embodiment of the invention, portions 401 and 404 represent the beginning and end of a wobble cycle of a wobbler transducer configuration whereas portions 402 and 405 represent the right and left image volume sides for such a wobbler transducer configuration. However, portions 401, 402, 404, and 405 may represent other image volume dataset boundaries, such as the most acute beam angles provided by a 2D matrix transducer array, the top boundary of a dataset, the bottom boundary of a dataset, etc.

Pictogram 412 is preferably displayed in conjunction with an image generated from a multi-dimensional dataset acquired using transducer 120 such that portion 401 is oriented to correspond with the side of transducer 120 having tool marker 121 thereon when the dataset was acquired, portion 402 is oriented to correspond with the side of transducer 120 having tool marker 123 thereon when the data set was acquired, portion 404 is oriented to correspond with the side of transducer 120 having tool marker 122 thereon when the dataset was acquired, and portion 405 is oriented to correspond with the side of transducer 120 having tool marker 124 thereon when the data set was acquired. Accordingly, a user may easily recognize the relationship between the orientation of the image with that of the transducer, and thus will intuitively be able to understand the relationship of the image to the physical world.

It should be appreciated that, by utilizing tool markers and thus image marker portions which are associated with orthogonal tool attributes, a user is readily and unambiguously able to appreciate the image orientation in 3D space. Accordingly, although the illustrated embodiment of pictogram 412 includes portions adapted to correspond with each of 4 tool markers, it should be appreciated that embodiments of the present invention may comprise a different configuration of pictogram. Preferred embodiments of the invention comprise a pictogram which include portions or attributes corresponding to at least 2 tool markers associated with orthogonal attributes of an imaging tool, such as different axes. As an example, an embodiment of the present invention may pictogram 412 having portions 401 and 402 corresponding to tool markers 121 and 123, themselves corresponding to a first axis of transducer 120 and tool marker 123 corresponding to a second axis of transducer 120. Additionally or alternatively, pictogram 412 of embodiments may provide express information, such as through the use of letters, numbers, and/or symbols on, in, or in association with the pictogram.

It should be appreciated that pictogram 412 of the illustrated embodiment represents the multi-dimensional dataset from which a corresponding image is generated. Accordingly, pictogram 412 of this embodiment is itself multi-dimensional (here, at least 3D). The use of pictograms provided in at least 3D further facilitates users understanding orientation, spatial, and/or relational information by providing robust relational information in an intuitive format. Moreover, where an image generated from a multi-dimensional dataset is generated in fewer dimensions than that of the dataset (e.g., generating a 2D MPR image from a 3D or 4D dataset), such pictograms facilitate an understanding of the space represented in the image and/or the orientation of the image.

Referring still to FIG. 4A. pictogram 412 of the illustrated embodiment includes portion 403 representing an image plane within the multi-dimensional dataset a currently selected or displayed image is associated with. For example, where 2D MPR images are generated from a 3D or 4D dataset (e.g., 2D cross-sectional images are generated from a 3D or 4D volume), portion 403 of pictogram 412 may show a user where within the dataset the currently displayed image is showing. As different MPR images are selected or displayed, portion 403 is preferably updated to properly reflect where within the dataset the image is showing, thereby providing a multi-dimensional pictogram of at least 4D. Although shown as a plane having an axis parallel to that of the long axis of transducer 120 in the illustrated embodiment, portion 403 may be provided in any orientation corresponding to a selected or displayed image, according to embodiments of the invention. Accordingly, portion 403 of embodiments may represent an image plane in any orientation within a dataset volume according to embodiments of the invention.

In order to simplify the information presented and/or the operation of imaging functionality, embodiments of the invention adopt image display conventions with respect to imaging provided for particular tasks, uses, situations, etc. and/or to provide images which are readily understood. For example, embodiments of the invention include providing an image coordinate system or other image display convention providing images in an consistent, intuitive orientation. As one exemplary configuration, embodiments providing ultrasound imaging utilize an image display convention to always orient shallow up and deep down, at least with respect to certain modes of operation particular functions or procedures, etc. For example, 3D volume rendered images and/or MPR images generated from a multi-dimensional volume dataset for use in an interventional procedure may be controlled to always display images in a shallow-up and deep-down orientation. Accordingly, irrespective of how a user controls movement and rotation with respect to each of the X, Y, and Z axes of an image plane within a dataset volume, the resulting image will be presented in a shallow-up and deep-down orientation.

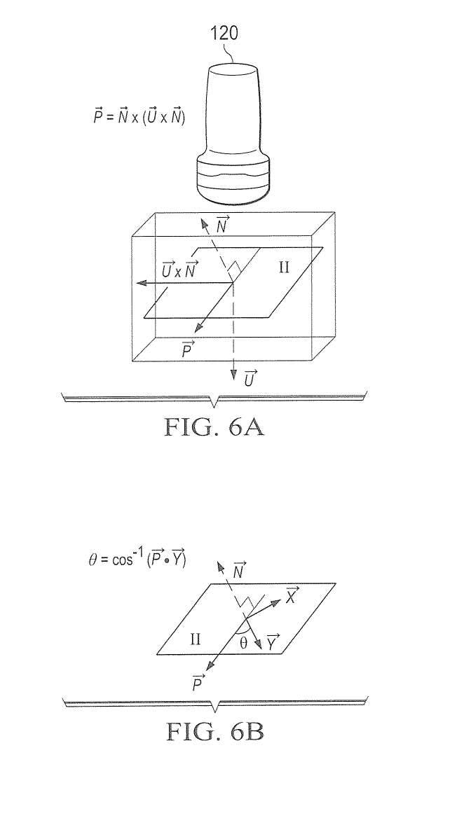

In operation according to embodiments of the invention: any arbitrary 2D cross-section image reconstructed from a 3D volume is displayed using image display conventions such that the most top part of the image corresponds to the shallowest depth and the most bottom part to the deepest depth. FIGS. 6A and 6B illustrate how such an image display convention may be implemented mathematically, wherein plane Π defines the reconstructed 2D cross-section, vector \vec{U} defines the shallow-deep direction (based on the transducer orientation), and vector \vec{P} is the projection of vector \vec{U} in plane Π. In this example it is assumed that, based on the provided 3D manipulation tools (rotation and translation around the 3 axes defining the three-dimensional coordinate system), the reconstructed 2D cross-section has a vertical axis defined by vector \vec{Y} and a horizontal axis defined by vector \vec{X}, whereas the normal to the 2D cross-section (and plane Π) is defined by vector \vec{N}. To display an arbitrary 2D cross-section with a vertical shallow-deep orientation according to embodiments of the invention, each produced 2D cross-section is rotated by angle θ, defined as the angle formed in plane Π between vectors \vec{P} and \vec{Y}. This angle can be computed based on the dot product of the vectors as described by the following equation:

θ=cos⁻¹(\vec{P}\vec{Y}) (1)

Where the projection vector \vec{P} can be computed by a sequence of cross products between vectors \vec{U} and \vec{N} as described by the following equation:

\vec{P}=\vec{N}×(\vec{U}×\vec{N}) (2)

It should be appreciated that the foregoing image display convention implementation, resulting in presentation of images in a shallow-up and deep-down orientation, is fundamentally different than the image presentation traditionally provided by 3D imaging systems. In particular, it is a fundamental aspect of most 3D imaging systems to facilitate a user positioning an object of interest, and thus a generated image containing the object of interest, in an orientation desired. This has been so because the object of interest is the focus of the image and is easily identified in any view thereof. However, the present inventors have discovered that, although containing an object of interest in a dataset volume, certain imaging operations are performed with respect to objects of interest which are not readily identified, such as due to unknown particulars of the object, unclear or obscured portions of the object, other objects appearing in the image, etc. Accordingly, adopting an image display convention which results in images always being presented in a shallow-up and deep-down orientation, regardless of the rotation and movement of the image plane within the dataset volume facilitates a user's identification of such an object of interest.

Image plane freedom limitations implemented according to embodiments of the invention may additionally or alternatively be utilized according to embodiments of the invention. Such image plane freedom limitations may provide restrictions with respect to numbers, types, and/or particular images which may be generated or displayed. For example, the ability to generate MPR images from a multi-dimensional image volume may be limited, at least in some modes of operation, to cross-sectional images generated along a particular axis, arc, etc. Although facilitating MPR images to be generated with respect to an entire multi-dimensional volume, embodiments of the invention restrict the degrees of freedom with respect to MPR image generation, such as when particular modes of operation are selected, when particular procedures are performed, etc. A preferred embodiment limits MPR image generation to a single degree of freedom, such as in a survey mode of operation. For example, embodiments of the invention provide a survey mode of operation wherein MPR image generation is limited to generating images in image planes corresponding to a method of acquiring a dataset volume. That is, embodiments of the invention may provide for MPR image sweeping through the dataset volume in accordance with an image data acquisition sweep used to generate the dataset volume. Such embodiments provide advantages in that the best image quality is provided because images are generated more directly from the collected image data (e.g., views need not be synthesized from the dataset volume) and the image sweep is likely to be intuitive to the user.

Consistent with the foregoing, according to an embodiment of the invention MPR images may be limited to those generated along a single arc, such as rotated about the long axis of FIGS. 1 and 4A to correspond with an image data collection sweep made using transducer 120. This image plane freedom limitation may be appreciated by its pictographic representation in pictogram 412 as shown in FIGS. 4A-4C. As previously discussed, portion 403 of pictogram 412 represents a current cross-sectional position of an MPR image within the dataset volume. In the pictogram of FIG. 4A, portion 403 is disposed approximately equidistant along the short axis from portions 401 and 404, indicating that an associated MPR image represents a corresponding center cross-section of the multi-dimensional image volume. However, in the pictogram of FIG. 4B, portion 403 has been rotated about the long axis towards portion 401, indicating that an associated MPR image represents a cross-section of the multi-dimensional image volume toward the front side of transducer 120. Similarly, in the pictogram of FIG. 4C, portion 403 has been rotated about the long axis towards portion 404, indicating that an associated MPR image represents a cross-section of the multi-dimensional image volume toward the back side of transducer 120.

Information may be provided in addition to the movement of portion 403, as shown in FIGS. 4A-4C, to aid a user in understanding the relative position of a generated or selected image within the volume dataset. For example, numerical data could be added in, to, or in association with pictogram 412 to facilitate a users' understanding. As one example, the numeral “0” provided with pictogram 412 may indicate that portion 403, and thus the associated image, is centered within the volume dataset, the numeral “+15” may indicate the last cross-section in the direction of portion 401, the numeral “−15” may indicate the last cross-section in the direction of portion 402, and numerals between indicating increments between these positions. Such an embodiment could be utilized to provide easy, organized, and complete access to 31 MPR cross-section images according to embodiments of the invention.

Although an image plane freedom limitation is implemented in the foregoing example such that portion 403, and thus the correspondingly rendered images, is only provided one degree of freedom (rotated about the long axis), a user is enabled to generate and/or select images which in the aggregate display the full dataset volume. For example, a user may survey the entire dataset volume by stepping through sequential cross-section images as portion 403 is incremented from portion 401 to portion 402. Such a survey provides information from which a user may easily identify one or more best images for a particular task, such as to facilitate semi-automated interventional apparatus image plane identification, selection, image generation, and/or display as shown and described in the above referenced patent application entitled “Systems and Methods to Identify Interventional Instruments.”

The use of image plane freedom limitations according to embodiments of the invention is preferably accompanied by a simplified user interface. For example, where the user is provided with one degree of freedom with respect to generation and/or display of images from a image volume dataset, embodiments of the invention may implement a relatively simple, preferably bidirectional, control, such as left and right buttons, spinner knob, single axis joystick, etc. as part of user interface 113 of system unit 110 (FIG. 1A) to facilitate user selection of images. A user may thus sweep through an acquired volume dataset by twisting a knob in the appropriate direction, pressing the appropriate directional button, displacing a joystick in the appropriate direction, etc. The user may stop at any desired image, such as to view the image, interact with the tool or image (e.g., zoom, pan, rotate, etc.), record or print the image, lock the plane, etc. The particular image selected or displayed may be represented by image marker 112, such as may comprise pictogram 412, to facilitate the user's interpretation of the image and/or understanding of the image's position within an image volume.

It should be appreciated that images presented according to embodiments of the present invention are not limited to a single volume rendered image or cross-section (e.g., MPR) image and corresponding image marker. For example, multiple views, representations, image forms, etc. of a dataset volume may be provided as shown in FIG. 5, wherein image 111 includes sub-images 511-513 and image marker 112. Sub-image 511 may, for example, comprise an A-plane view of the dataset volume (e.g., an image plane selected to show the interventional instrument), sub-image 512 may comprise a B-plane view (e.g., a vertical cross-sectional plane orthogonal to the A-plane view), and sub-image 513 may comprise a C-plane view (e.g., a horizontal cross-sectional plane orthogonal to the A-plane view). Of course, additional or alternative sub-images may be displayed according to embodiments of the invention, such as to provide an image for the survey mode described herein, a volume rendered image, etc.

Although embodiments have been described herein with respect to use of system 100 for interventional procedures, it should be appreciated that systems adapted according to the concepts of the present invention may be for any number of uses. For example, the tool and image relational features, the image coordinate system conventions, and the image volume survey features of embodiments of the invention may be particularly useful with respect to imaging less recognizable structure, such as nerves, blood vessels, intestines, etc. Moreover, embodiments of the invention may be used with respect to any number of targets and media, such as fluids, containers and vessels, soil, etc., and thus are not limited to the exemplary human body.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method comprising:
providing a first tool marker in association with a first selected aspect of a tool used for imaging;
providing a second tool marker in association with a second selected aspect of said tool, wherein said first tool marker and said second tool marker are associated with orthogonal attributes of said tool;
rendering an image using information provided by said tool; and
providing image marking in association with said image to provide correlation between an orientation of said tool and said image, wherein said image marking comprises a first aspect corresponding to said first tool marker and a second aspect corresponding to said second tool marker.

2. The method of claim 1, wherein said information provided by said tool comprises a multi-dimensional dataset having at least spatial 3 dimensions.

3. The method of claim 2, wherein said information provided by said tool comprises a multi-dimensional dataset having at least 4 dimensions.

4. The method of claim 1, wherein said orthogonal attributes comprise different axes.

5. The method of claim 1, wherein said tool comprises a transducer used to collect imaging data.

6. The method of claim 5, wherein said transducer comprises an ultrasound transducer and said image comprises an ultrasound image.

7. The method of claim 1, wherein said image comprises a volume rendered image and said first aspect and said second aspect of said image marking correspond to different portions of a volume of said volume rendered image.

8. The method of claim 1, wherein said image comprises a cross-sectional view of a volume and said first aspect and said second aspect of said image marking correspond to different portions of said volume.

9. The method of claim 8, wherein said image marking comprises:
an indicator of a position within said volume said cross-sectional view shows.

10. The method of claim 1, wherein said image marking comprises:
a pictogram.

11. The method of claim 10, wherein said pictogram provides a graphical representation of a volume of which said image is representative.

12. The method of claim 10, wherein said pictogram comprises:
a first side having said first aspect; and
a second side having said second aspect, wherein said first side is a side of said volume corresponding to said first aspect of said tool and said second side is a side of said volume corresponding to said second aspect of said tool.

13. The method of claim 12, wherein said first aspect of said tool comprises a first side of said tool and said second aspect of said tool comprises a second side of said tool.

14. The method of claim 10, wherein said pictogram comprises:
an indicator corresponding to an area within an image volume from which a currently selected image portion is displayed.

15. The method of claim 14, wherein said indicator comprises:
an image plane, said image plane being movable within said pictogram to indicate said area within an image volume from which a currently selected image portion is displayed.

16. The method of claim 1, further comprising:
implementing at least one selected image display convention with respect to said image, said selected image display convention always presenting said image in a particular orientation irrespective of an orientation of an image plane of said image within a volume being imaged.

17. The method of claim 16, wherein said at least one selected image display convention is selected as a function of at least one of a procedure being performed and an image mode being used.

18. The method of claim 1, further comprising:
implementing at least one selected image plane freedom limitation with respect to said image, wherein said at least one selected image plane freedom limitation restricts degrees of freedom with respect to generating cross-sectional views of an image volume to a single degree of freedom.

19. The method of claim 18, further comprising:
providing a survey of said image volume by navigating said single degree of freedom.

20. The method of claim 1, further comprising:
implementing an image display convention for orientation of images, including said image, generated from at least a three-dimensional dataset to a single preselected orientation.

21. The method of claim 20, wherein said preselected orientation comprises a shallow up and deep down image orientation.

22. A method comprising:
generating a m-dimensional image volume dataset using information provided by an imaging tool;
rendering a n-dimensional image from said m-dimensional image volume dataset, wherein n<m; and
restricting degrees of freedom with respect to said generating said n-dimensional image to a degree of freedom along a dimension of said m-dimensional image volume dataset which is not a dimension of said n-dimensional image.

23. The method of claim 22, wherein said restricting degrees of freedom with respect to said generating said n-dimensional image comprises:
restricting generation of said n-dimensional image to image planes corresponding to an information acquisition technique used by said imaging tool.

24. The method of claim 22, wherein m is at least 3 and n is at least 2.

25. The method of claim 22, said restricting degrees of freedom with respect to generating said n-dimensional image comprises:
restricting said degrees of freedom to a single degree of freedom.

26. The method of claim 24, wherein said restricting degrees of freedom with respect to said generating said n-dimensional image further comprises:
providing one-dimensional control for controlling generation of said n-dimensional image.

27. The method of claim 25, wherein said image comprises a cross-sectional image of said m-dimensional image volume dataset and said single degree of freedom is orthogonal to an image plane of said cross-sectional image.

28. The method of claim 22, further comprising:
providing image marking in association with said image to provide correlation between an orientation of said tool and said image.

29. The method of claim 28, further comprising:
providing a first tool marker in association with a first aspect of said tool; and
providing a second tool marker in association with a second aspect of said tool, wherein said image marking comprises a first aspect corresponding to said first tool marker and a second aspect corresponding to said second tool marker, wherein said first tool marker and said second tool marker are associated with orthogonal attributes of said tool.

30. The method of claim 29, wherein said first aspect of said tool comprises a first side of said tool and said second aspect of said tool comprises a second side of said tool.

31. The method of claim 28, wherein said image marking comprises:
a pictogram.

32. The method of claim 31, wherein said pictogram provides a graphical representation of said m-dimensional image volume.

33. The method of claim 31, wherein said pictogram comprises:
an indicator corresponding to an area within said m-dimensional image volume said rendered image represents.

34. The method of claim 33, further comprising:
providing an image plane indicator in said pictogram, said image plane indicating an area of said image a corresponding image represents; and
moving said image plane within said pictogram to correspond with an area of a currently rendered image.

35. The method of claim 22, further comprising:
providing a survey of said m-dimensional image volume dataset by navigating said degree of freedom.

36. The method of claim 22, further comprising:
implementing an image display convention for orientation of images, including said n-dimensional image, generated from said m-dimensional image volume dataset to provide display of said images in a single preselected orientation.

37. The method of claim 36, wherein said preselected orientation comprises a shallow up and deep down image orientation.

38. A method comprising:
generating an m-dimensional image volume dataset using information provided by an imaging tool;
rendering a n-dimensional image from said m-dimensional image volume dataset, wherein n<m, and wherein an image plane of said n-dimensional image has a location and orientation within said m-dimensional image volume; and
implementing an image display convention for display orientation of said n-dimensional image to provide display of said n-dimensional image in a single preselected orientation, said display orientation providing said display orientation regardless of a particular said location and orientation of said n-dimensional image within said m-dimensional image volume.

39. The method of claim 38, wherein said preselected orientation comprises a shallow up and deep down image orientation.

40. The method of claim 38, further comprising:
implementing at least one selected image plane freedom limitation with respect to said n-dimensional image.

41. The method of claim 40, wherein said at least one selected image plane freedom limitation is selected as a function of at least one of a procedure being performed and an image mode being used.

42. The method of claim 40, wherein said implementing said at least one selected image plane freedom limitation comprises:
restricting degrees of freedom with respect to generating cross-sectional views of said m-dimensional image volume to a single degree of freedom.

43. The method of claim 42, further comprising:
providing a survey of said m-dimensional image volume by navigating said single degree of freedom.

44. A system comprising:
an imaging tool operable to collect image data;
an imaging processor operable to generate an image volume dataset using said image data and to render an image from said image volume dataset;
a display operable to display said image and an image marking in association with said image, wherein said image marking provides correlation between at least two aspects of said tool and said image.

45. The system of claim 44, wherein said at least two aspects comprise a first side and a second side.

46. The system of claim 44, wherein said tool comprises:
a first tool marker in association with a first aspect of said tool; and
a second tool marker in association with a second aspect of said tool, wherein said image marking comprises a first aspect corresponding to said first tool marker and a second aspect corresponding to said second tool marker.

47. The system of claim 44, wherein said image marking comprises:
a pictogram.

48. The system of claim 47, wherein said pictogram provides a graphical representation of said image volume dataset.

49. The system of claim 47, wherein said pictogram comprises:
an indicator corresponding to an area within an image volume dataset said displayed image represents.

50. The system of claim 49, wherein said indicator comprises:
an image plane indicator, said image plane indicator being movable within said pictogram to correspond with an area of a currently displayed image.

51. The system of claim 49, further comprising:
a bidirectional user interface for navigating said indicator through said pictogram and correspondingly selecting images throughout said image volume dataset.

52. The system of claim 44, wherein said imaging processor limits degrees of freedom with respect to said image volume dataset to a single degree of freedom.

53. The system of claim 52, wherein said single degree of freedom comprises a degree of freedom which is orthogonal to an image plane of said displayed image.

54. The system of claim 44, wherein said imaging processor implements an image display convention for display orientation of said image to provide display of said image in a single preselected orientation, said display orientation providing said display orientation regardless of a particular said location and orientation of said image within said image volume.

55. The system of claim 44, wherein said system comprises:
an ultrasound imaging system.

56. The system of claim 55, wherein said imaging processor comprises:
an ultrasound system unit processor.

57. The system of claim 56, wherein said imaging tool comprises:
an ultrasound transducer.

58. The system of claim 57, wherein said ultrasound transducer comprises a transducer selected from the group consisting of:
a piezoelectric transducer;
a capacitive micro-machined ultrasonic transducer (CMUT);
a piezoelectric micro-machined ultrasonic transducer (PMUT);
a wobbler transducer;
a 1D matrix array transducer;
a 1.5D matrix array transducer;
a 1.75D matrix array transducer;
a 2D matrix array transducer;
a linear array transducer; and
a curved array transducer.

59. The system of claim 44, wherein said system comprises:
front-end circuitry coupled to said imaging tool;
a mid-processor disposed in a signal path between said front-end circuitry and said image processor and operable to provide signal and image processing with respect to said image data; and
a back-end processor coupled to said signal path, said back-end processor comprising said imaging processor.

60. A system comprising:
an imaging tool operable to collect image data;
an imaging processor operable to generate an image volume dataset using said image data and to render images from said image volume dataset;
a one-dimensional control for controlling generation of said images, wherein said one-dimensional control restricts degrees of freedom with respect to said generating said image to a degree of freedom along a dimension of said image volume dataset which is not a dimension of said images.

61. The system of claim 60, wherein said one-dimensional control controls generation of said images in image planes corresponding to an information acquisition technique used by said imaging tool.

62. The system of claim 60, wherein said one-dimensional control comprises:
a bidirectional user interface for navigating image generation through said image volume dataset.

63. The system of claim 60, further comprising:
a display operable to display said images and an image marking in association with said images, wherein said image marking provides correlation between at least two aspects of said tool and said images.

64. The system of claim 63, wherein said tool comprises:
a first tool marker in association with a first aspect of said tool; and
a second tool marker in association with a second aspect of said tool, wherein said image marking comprises a first aspect corresponding to said first tool marker and a second aspect corresponding to said second tool marker.

65. The system of claim 63, wherein said image marking comprises:
a pictogram.

66. The system of claim 65, wherein said pictogram comprises:
an indicator corresponding to an area within said image volume dataset said displayed image represents.

67. The system of claim 66, wherein said indicator comprises:
an image plane indicator, said image plane indicator being movable within said pictogram to correspond with an area of a currently displayed image.

68. The system of claim 60, wherein said system comprises:
an ultrasound imaging system.

69. The system of claim 68, wherein said imaging processor comprises:
an ultrasound system unit processor.

70. The system of claim 69, wherein said imaging tool comprises:
an ultrasound transducer.

71. The system of claim 70, wherein said ultrasound transducer comprises a transducer selected from the group consisting of:
a piezoelectric transducer;
a capacitive micro-machined ultrasonic transducer (CMUT);
a piezoelectric micro-machined ultrasonic transducer (PMUT);
a wobbler transducer:
a 1D matrix array transducer;
a 1.5D matrix array transducer;
a 1.75D matrix array transducer;
a 2D matrix array transducer;
a linear array transducer; and
a curved array transducer.

72. The system of claim 60, wherein said system comprises:
front-end circuitry coupled to said imaging tool;
a mid-processor disposed in a signal path between said front-end circuitry and said image processor and operable to provide signal and image processing with respect to said image data; and
a back-end processor coupled to said signal path, said back-end processor comprising said imaging processor.
